(19) United States
(12) Patent Application Publication
(10) Pub. No.: US 2025/0389715 A1
(43) Pub. Date: Dec. 25, 2025

(54) POINT-OF-CARE BIOASSAY SYSTEMS AND METHODS OF MAKING AND USING THE SAME

(71) Applicant: AmplifiDx, Inc., Newark, CA (US)

(72) Inventors: Michael Reber WHITE, La Habra Heights, CA (US); Omar ABDALLAH, Los Angeles, CA (US); Gordon Fox GRAJEK, Orange, CA (US); Shoulian DONG, Mountain View, CA (US); Nancy SCHOENBRUNNER, Charlestown, MA (US)

(73) Assignee: AMPLIFIDX, INC., Newark, CA (US)

(21) Appl. No.: 19/248,112

(22) Filed: Jun. 24, 2025

Related U.S. Application Data
(60) Provisional application No. 63/663,634, filed on Jun. 24, 2024.

(51) Int. Cl. G01N 33/52; G01N 21/64

(52) U.S. Cl. CPC G01N 33/525; G01N 21/6428; G01N 2021/6421

(57) ABSTRACT

Devices and methods for performing rapid low-cost bioassays, including some embodiments for multiplexed detection of multiple amplified biomolecules for identifying one or more diseases, disorders, or infections, are disclosed herein. Such methods can include isolating a first biomolecule in a first detection chamber, isolating a second biomolecule in a second detection chamber, illuminating the first detection chamber with a first light source, and illuminating the second detection chamber with a second light source, where the illumination induces the production of an emitted light signal.

### CROSS-REFERENCE TO RELATED PATENT APPLICATIONS

This application claims priority to U.S. Appl. Ser. No. 63/663,634, filed Jun. 24, 2024, entitled “Point-Of-Care Bioassay Systems and Methods of Making and Using the Same,” which patent application is commonly owned by the owner of the present invention. This patent application is incorporated herein in its entirety.

### BACKGROUND

In many medical emergencies, such as the sudden spread of a highly contagious infectious agent (e.g., COVID19), implementing widespread testing with accurate, easy-to-use rapid and low-cost assays is paramount for assessing and controlling its impact. Real-time PCR tests are highly-sensitive and accurate for assessing viral loads. However, these tests have the disadvantage of requiring high sample preparation time, reagent handling protocols and personnel with specialized training. While many companies have launched assay systems that allow for point of care testing, typically they require assay cartridges and instrumentation that are bulky, complex and costly.

Accordingly, there is a need, especially for medical applications in resource poor settings, for simpler and less costly devices for rapid and effective testing in populations exposed to highly contagious viral diseases.

### SUMMARY OF THE DISCLOSURE

The present disclosure is directed to methods, systems and self-metering cartridges, including microfluidic devices, for implementing rapid low-cost point-of-care bioassays, including nucleic acid-based bioassays. In one embodiment, the disclosure describes a cartridge for performing a multiplexed bioassay on a biological sample to determine the presence or quantity of one or more biomolecules, such as one or more polynucleotides. The cartridge may be associated with an appliance that is a multi-use device that provide one or more thermal sources, negative and positive pressure sources, light sources, optical detectors, optical filters, and optical occluders. In some embodiments, the cartridge comprises a lysis buffer chamber, a sample chamber, a first detection chamber, and a second detection chamber.

In some embodiments, the disclosure features a method of operating the system comprises amplifying a biomolecule, and associating the amplified biomolecule with a fluorescent label that is capable of optical detection, such as a fluorophore. In some embodiments, the fluorescent label can be covalently or non-covalently bound to the amplified biomolecule. In some embodiments, the fluorescent label is covalently bound to a probe which, in turn, is non-covalently bound to the amplified biomolecule.

In some embodiments, the disclosure features a method comprises isolating a first amplified biomolecule in a first detection chamber, wherein the first amplified biomolecule comprises fluorophore 1A; isolating a second amplified biomolecule in a second detection chamber, wherein the second amplified biomolecule comprises fluorophore 2A; illuminating the first detection chamber with a light source, wherein illuminating said first detection chamber induces fluorophore 1A of the first amplified biomolecule to produce emitted light signal 1A; and illuminating the second detection chamber with a light source, wherein illuminating said second detection chamber induces fluorophore 2A of the second amplified biomolecule to produce emitted light signal 2A. In some embodiments, the light source that illuminates the first and second detection chambers is the same.

In some embodiments, the disclosure features a method in which a light source emits at least one wavelength of light, which is directed towards the first and second detection chambers. In one embodiment, the method further comprising positioning an optical occluder in a first position to block the second detection chamber from at least a portion of the first wavelength of light, wherein the first detection chamber is exposed to the first wavelength of light and fluorophore 1A of the first biomolecule produces emitted light signal 1A. In one embodiment, the method further comprises positioning the optical occluder in a second position to block the first detection chamber from at least a portion of the first wavelength of light, wherein the second detection chamber is exposed to the first wavelength of light and fluorophore 2A of the second biomolecule produces emitted light signal 2A. In some embodiments, emitted light signal 1A and/or 1B are filtered to produce at least one filtered light signal that is detected by the optical detector.

In some embodiments, the disclosure features an optical detection system. The optical detection system can include a lysis buffer chamber containing lysis buffer. The optical detection system can include a sample chamber in fluid communication with the lysis buffer chamber. The optical detection system can include a first detection chamber containing a first set of assay reagents. The first detection chamber can be in fluid communication with the sample chamber and a first port. The optical detection system can include a second detection chamber containing a second set of assay reagents. The second detection chamber can be in fluid communication with the sample chamber and a second port. The optical detection chamber can include a pump in fluid communication with the first port and the second port, wherein the pump is operatively configured to selectively apply a negative pressure and a positive pressure to said first port and said second port. The optical detection chamber can include a first light source positioned to emit a first wavelength towards the first detection chamber and the second detection chamber. The optical detection chamber can include an optical occluder operatively configured to be positioned in a first position to occlude the first detection chamber and expose the second detection chamber, and a second position to expose the first detection chamber and occlude the second detection chamber. The optical detection chamber can include a first optical filter. The optical detection chamber can include an optical detector.

In some embodiments, the disclosure features a method including introducing a biological sample into a sample chamber. The method can include allowing a lysis buffer to pass from a lysis buffer chamber to the sample chamber. The method can include allowing the lysis buffer to mix with the biological sample to create a biological mixture. The method can include engaging a pump to apply a negative pressure to move a first portion of the biological mixture to a first detection chamber and a second portion of the biological mixture from the sample chamber to a second detection chamber. The method can include allowing the first portion of the biological mixture to react with a first set of assay reagents in the first detection chamber to create a first amplified biomolecule bound to fluorophore 1A. The method can include allowing the second portion of the biological mixture to react with a second set of assay reagents in the second detection chamber to create a second amplified biomolecule bound to fluorophore 2A. The method can include engaging the first light source to emit a first wavelength of light toward the first detection chamber and the second detection chamber. The method can include positioning an optical occluder in a second position to block at least a portion the second detection chamber from the first wavelength of light emitted by the first light source, and expose the first detection chamber to the first wavelength of light, wherein fluorophore 1A of the first biomolecule produces emitted light signal 1A. The method can include filtering emitted light signal 1A from the first fluorophore through the first optical filter to produce filtered light signal 1A. The method can include detecting filtered light signal 1A at an optical detector. The method can include positioning the optical occluder to a first position to expose the second detection chamber to the first wavelength of light and block at least a portion of the first detection chamber from the first wavelength of light, wherein the second fluorophore of the second biomolecule produces emitted light signal 2A. The method can include filtering emitted light signal 2A from the second fluorophore through the first optical filter to produce filtered light signal 2A. The method can include detecting filtered light signal 2A at the optical detector.

### BRIEF DESCRIPTION OF THE DRAWINGS

Other advantages of the present disclosure will be apparent from the following detailed description of the disclosure in conjunction with embodiments as illustrated in the accompanying drawings, in which:

FIG. 1 illustrates diagrammatically the basic design of an embodiment of a cartridge body, in accordance with certain embodiments of the present disclosure.

FIG. 2 provides an exploded view of an exemplary cartridge 3 with additional components added to cartridge body 1, in accordance with certain embodiments of the present disclosure.

FIG. 3 provides an exploded view of an appliance with cartridge 3 for reading after a biological sample has been received in sample chamber 6, in accordance with certain embodiments of the present disclosure.

FIG. 4 provides a top view of a cartridge engaged with the optics module of the appliance, in accordance with certain embodiments of the present disclosure.

FIG. 5 provides for multiple embodiments in which the biological sample in sample chamber 6 is mixed and then drawn into the detection chambers via negative and positive pressure, respectively, in accordance with certain embodiments of the present disclosure.

FIG. 6 demonstrates the movement of biological samples into the detection chambers, in accordance with certain embodiments of the present disclosure.

FIG. 7 presents an assay workflow for an exemplary diagnostic cartridge, in accordance with certain embodiments of the present disclosure.

### NOTATION AND NOMENCLATURE

Various terms are used to refer to particular system components. Different companies may refer to a component by different names—this document does not intend to distinguish between components that differ in name but not function. In the following discussion and in the claims, the terms “including” and “comprising” are used in an open-ended fashion, and thus should be interpreted to mean “including, but not limited to” Also, the term “couple” or “couples” is intended to mean either an indirect or a direct connection. Thus, if a first device couples to a second device, that connection may be through a direct connection or through an indirect connection via other devices and connections.

The terminology used herein is for the purpose of describing particular example embodiments only, and is not intended to be limiting. Following long-standing patent law convention, the terms “a” and “an” mean “one or more” when used in this application, including the claims.

As used herein, the singular forms “a,” “an,” and “the” may be intended to include the plural forms as well, unless the context clearly indicates otherwise. The method steps, processes, and operations described herein are not to be construed as necessarily requiring their performance in the particular order discussed or illustrated, unless specifically identified as an order of performance. It is also to be understood that additional or alternative steps may be employed.

The terms first, second, third, etc. may be used herein to describe various elements, components, regions, layers and/or sections; however, these elements, components, regions, layers and/or sections should not be limited by these terms. These terms may be only used to distinguish one element, component, region, layer, or section from another region, layer, or section. Terms such as “first,” “second,” and other numerical terms, when used herein, do not imply a sequence or order unless clearly indicated by the context. Thus, a first element, component, region, layer, or section discussed below could be termed a second element, component, region, layer, or section without departing from the teachings of the example embodiments. The phrase “at least one of,” when used with a list of items, means that different combinations of one or more of the listed items may be used, and only one item in the list may be needed. As used herein, the term “and/or” when used in the context of a listing of entities, refers to the entities being present singly or in combination. Thus, for example, the phrase “A, B, C, and/or D” includes A, B, C, and D individually, but also includes any and all combinations and subcombinations of A, B, C, and D. Accordingly, as an example, “at least one of: A, B, and C” includes any of the following combinations: A, B, C, A and B, A and C, B and C, and A and B and C. In another example, the phrase “one or more” when used with a list of items means there may be one item or any suitable number of items exceeding one.

Spatially relative terms, such as “inner,” “outer,” “beneath,” “below,” “lower,” “above,” “upper,” “top,” “bottom,” and the like, may be used herein. These spatially relative terms can be used for ease of description to describe one element's or feature's relationship to another element(s) or feature(s) as illustrated in the figures. The spatially relative terms may also be intended to encompass different orientations of the device in use, or operation, in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptions used herein interpreted accordingly.

Unless otherwise indicated, all numbers expressing quantities of ingredients, reaction conditions, and so forth used in the specification are to be understood as being modified in all instances by the term “about.” Accordingly, unless indicated to the contrary, the numerical parameters set forth in this specification are approximations that can vary depending upon the desired properties sought to be obtained by the presently disclosed subject matter.

“Amplicon” means the product of a polynucleotide amplification reaction; that is, a clonal population of polynucleotides, which may be single stranded or double stranded, which are replicated from one or more starting sequences. “Amplifying” means producing an amplicon by carrying out an amplification reaction. The one or more starting sequences may be one or more copies of the same sequence, or they may be a mixture of different sequences. Preferably, amplicons are formed by the amplification of a single starting sequence.

Amplicons may be produced by a variety of amplification reactions whose products comprise replicates of the one or more starting, or target, nucleic acids. In one aspect, amplification reactions producing amplicons are “template-driven” in that base pairing of reactants, either nucleotides or oligonucleotides, have complements in a template polynucleotide that are required for the creation of reaction products. In one aspect, template-driven reactions are primer extensions with a nucleic acid polymerase or oligonucleotide ligations with a nucleic acid ligase. Such reactions include, but are not limited to, polymerase chain reactions (PCRs), linear polymerase reactions, nucleic acid sequence-based amplification (NASBAs), and rolling circle amplifications. In one aspect, amplicons disclosed herein are produced by PCRs.

An amplification reaction may be a “real-time” amplification if a detection chemistry is available that permits a reaction product to be measured as the amplification reaction progresses, e.g., “real-time PCR” described below, or “real-time NASBA”. As used herein, the term “amplifying” means performing an amplification reaction. A “reaction mixture” means a solution containing all the necessary reactants for performing a reaction, which may include, but not be limited to, buffering agents to maintain pH at a selected level during a reaction, salts, co-factors, scavengers, and the like.

“Dried reagents” mean assay reagents, such as buffers, salts, active compounds, such as enzymes, co-factors, and the like, or binding compounds, such as antibodies, aptamers, or the like, that are provided in a dehydrated formulation for the purpose of improved shelf-life, ease of transport and handling, improved storage, and the like. The nature, composition, and method of producing dried reagents vary widely. Dried reagents include, but are not limited to, solid and/or semi-solid particulates, powders, tablets, crystals, capsules and the like, that are manufactured in a variety of ways. In one aspect, dried reagents are lyophilized particulates. Lyophilized particulates may have uniform compositions, wherein each particulate has the same composition, or they may have different compositions, such that two or more different kinds of lyophilized particulates having different compositions are mixed together. Lyophilized particulates can contain reagents for all or part of a wide variety of assays and biochemical reactions, including immunoassays, enzyme-based assays, enzyme substrate assays, and the like. In one aspect, a lyophilized particulate of the present disclosure comprises an excipient and at least one reagent of an assay. Lyophilized particulates may be manufactured in predetermined sizes and shapes, which may be determined by the type of assay being conducted, desired reaction volume, desired speed of dissolution, and the like. In some embodiments, lyophilized particulates are provided in a size such that they are mobile within whatever chamber they are disposed in.

Dried reagents may include excipients, which are usually inert substances added to a material in order to confer a suitable consistency or form to the material. A large number of excipients exist and can comprise a number of different chemical structures. Examples of excipients, which may be used in the present invention, include carbohydrates, such as sucrose, glucose, trehalose, melezitose, dextran, and mannitol; proteins such as BSA, gelatin, and collagen; and polymers such as PEG and polyvinyl pyrrolidone (PVP). The total amount of excipient in the lyophilized particulate may comprise either single or multiple compounds. In some embodiments, the type of excipient is a factor in controlling the amount of hygroscopy of a dried reagent. Lowering hygroscopy can enhance the dried reagent's integrity and cryoprotectant abilities. However, removing all water from such a composition would have deleterious effects on those reaction components, proteins for example, that require certain amounts of bound water in order to maintain proper conformations. Accordingly, in certain embodiments, the “lyophilized” or “dried” reagents described herein may comprise at least some water, such as about 0.001 to about 5 wt. %, about 0.01 to about 3 wt. %, or even about 0.1 to about 1.5 wt. % of bound water.

“Isothermal amplification” in reference to an assay to detect or quantify a target nucleic acid or polynucleotide means a method of replicating a target nucleic acid without a requirement of thermal cycling. That is, without a requirement of subjecting a reaction mixture to cycles of different temperatures in order to melt target nucleic acid strands, anneal primers and provide for extension conditions for a DNA polymerase. An isothermal amplification is typically performed at a predetermined temperature.

“Microfluidics” device or “nanofluidics” device, used interchangeably herein, each means an integrated system for capturing, moving, mixing, dispensing or analyzing small volumes of fluid, including samples (which, in turn, may contain or comprise cellular or molecular analytes of interest), reagents, dilutants, buffers, or the like. Generally, reference to “microfluidics” and “nanofluidics” denotes different scales in the size of devices and volumes of fluids handled. In some embodiments, features of a microfluidic device have cross-sectional dimensions of less than a few hundred square micrometers and have passages, or channels, with capillary dimensions, e.g., having maximal cross-sectional dimensions of from about 1-2 mm to about 0.1 μm. In some embodiments, microfluidics devices have volume capacities in the range of from 100 μL to a few nL, e.g. 10-100 nL or in the range of from 100 μL to 1 μL. Dimensions of corresponding features, or structures, in nanofluidics devices are typically from 1 to 3 orders of magnitude less than those for microfluidics devices. In some embodiments, microfluidic or nanofluidic devices have one or more chambers, ports, and channels that are interconnected and in fluid communication and that are designed for carrying out one or more analytical reactions or processes, either alone or in cooperation with an appliance or instrument that provides support functions, such as sample introduction, fluid and/or reagent driving means, such as positive or negative pressure, acoustical energy, or the like, temperature control, detection systems, data collection and/or integration systems, and the like. In some embodiments, microfluidics and nanofluidics devices may further include valves, pumps, filters and specialized functional coatings on interior walls, e.g., to prevent adsorption of sample components or reactants, facilitate reagent movement by electroosmosis, or the like. Such devices may be fabricated as an integrated device in a solid substrate, which may be glass, plastic, or other solid polymeric materials, and may have a planar format for ease of detecting and monitoring sample and reagent movement, especially via optical or electrochemical methods. In some embodiments, such devices are disposable after a single use. In some embodiments, microfluidic and nanofluidic devices include devices that form and control the movement, mixing, dispensing and analysis of droplets, such as, aqueous droplets immersed in an immiscible fluid, such as a light oil. The fabrication and operation of microfluidic and nanofluidic devices are well-known in the art.

“NASBA” or “Nucleic acid sequence-based amplification” is an amplification reaction based on the simultaneous activity of a reverse transcriptase (usually avian myeloblastosis virus (AMV) reverse transcriptase), an RNase H, and an RNA polymerase (usually T7 RNA polymerase) that uses two oligonucleotide primers, and which under conventional conditions can amplify a target sequence by a factor in the range of 109 to 1012 in 90 to 120 minutes. In a NASBA reaction, nucleic acids are a template for the amplification reaction only if they are single stranded and contain a primer binding site. Because NASBA is isothermal (usually carried out at 41° C. with the above enzymes), specific amplification of single stranded RNA may be accomplished if denaturation of double stranded DNA is prevented in the sample preparation procedure. That is, it is possible to detect a single stranded RNA target in a double stranded DNA background without getting false positive results caused by complex genomic DNA, in contrast with other techniques, such as RT-PCR. By using fluorescent indicators compatible with the reaction, such as molecular beacons, NASBAs may be carried out with real-time detection of the amplicon. Molecular beacons are stem-and-loop-structured oligonucleotides with a fluorescent label at one end and a quencher at the other end, e.g. 5′-fluorescein and 3′-(4-(dimethylamino)phenyl)azo) benzoic acid (i.e., 3′-DABCYL), as disclosed by Tyagi and Kramer (cited above). An exemplary molecular beacon may have complementary stem strands of six nucleotides, e.g. 4 G's or C's and 2 A's or T's, and a target-specific loop of about 20 nucleotides, so that the molecular beacon can form a stable hybrid with a target sequence at reaction temperature, e.g. 41° C. A typical NASBA reaction mix is 80 mM Tris-HCl [pH 8.5], 24 mM MgCl2, 140 mM KCl, 1.0 mM DTT, 2.0 mM of each dNTP, 4.0 mM each of ATP, UTP and CTP, 3.0 mM GTP, and 1.0 mM ITP in 30% DMSO. Primer concentration is 0.1 μM and molecular beacon concentration is 40 nM. Enzyme mix is 375 sorbitol, 2.1 μg BSA, 0.08 U RNase H, 32 U T7 RNA polymerase, and 6.4 U AMV reverse transcriptase. A reaction may comprise 5 μL sample, 10 μL NASBA reaction mix, and 5 μL enzyme mix, for a total reaction volume of 20 μL. Nested NASBA reactions are carried out similarly to nested PCRs; namely, the amplicon of a first NASBA reaction becomes the sample for a second NASBA reaction using a new set of primers, at least one of which binds to an interior location of the first amplicon.

“Polynucleotide” or “oligonucleotide” are used interchangeably and each mean a linear polymer of nucleotide monomers or analogs thereof. Monomers making up polynucleotides and oligonucleotides are capable of specifically binding to a natural polynucleotide by way of a regular pattern of monomer-to-monomer interactions, such as Watson-Crick type of base pairing, base stacking, Hoogsteen or reverse Hoogsteen types of base pairing, or the like. Such monomers and their internucleosidic linkages may be naturally occurring or may be analogs thereof, e.g. naturally occurring or non-naturally occurring analogs. Non-naturally occurring analogs may include PNAs, phosphorothioate internucleosidic linkages, bases containing linking groups permitting the attachment of labels, such as fluorophores, or haptens, and the like. Whenever the use of an oligonucleotide or polynucleotide requires enzymatic processing, such as extension by a polymerase, ligation by a ligase, or the like, oligonucleotides or polynucleotides in those instances would not contain certain analogs of internucleosidic linkages, sugar moieties, or bases at any or some positions. Polynucleotides typically range in size from a few monomeric units, e.g., 5-40, when they are usually referred to as “oligonucleotides,” to several thousand monomeric units. Whenever a polynucleotide or oligonucleotide is represented by a sequence of letters (upper or lower case), such as “ATGCCTG,” it will be understood that the nucleotides are in 5′→3′ order from left to right and that “A” denotes deoxyadenosine, “C” denotes deoxycytidine, “G” denotes deoxyguanosine, and “T” denotes thymidine, “I” denotes deoxyinosine, “U” denotes uridine, unless otherwise indicated or obvious from context. Unless otherwise noted the terminology and atom numbering conventions will follow those disclosed in Strachan and Read, Human Molecular Genetics 2 (Wiley-Liss, New York, 1999). Usually, polynucleotides comprise the four natural nucleosides (e.g., deoxyadenosine, deoxycytidine, deoxyguanosine, deoxythymidine for DNA or their ribose counterparts for RNA) linked by phosphodiester linkages; however, they may also comprise non-natural nucleotide analogs, e.g. including modified bases, sugars, or internucleosidic linkages. Where an enzyme has specific oligonucleotide or polynucleotide substrate requirements for activity, e.g., single stranded DNA, RNA/DNA duplex, or the like, an appropriate composition for the oligonucleotide or polynucleotide substrates may then be selected. Likewise, the oligonucleotide and polynucleotide may refer to either a single stranded form or a double stranded form (i.e., duplexes of an oligonucleotide or polynucleotide and its respective complement).

“Primer” means an oligonucleotide, either natural or synthetic that is capable, upon forming a duplex with a polynucleotide template, of acting as a point of initiation of nucleic acid synthesis and being extended from its 3′ end along the template so that an extended duplex is formed. Extension of a primer is usually carried out with a nucleic acid polymerase, such as a DNA or RNA polymerase. The sequence of nucleotides added in the extension process is determined by the sequence of the template polynucleotide. Usually, primers are extended by a DNA polymerase. Primers usually have a length in the range of from 14 to 40 nucleotides, or in the range of from 18 to 36 nucleotides. Primers are employed in a variety of nucleic amplification reactions, for example, linear amplification reactions using a single primer, or polymerase chain reactions, employing two or more primers.

“Polymerase chain reaction,” or “PCR,” means a reaction for the in vitro amplification of specific DNA sequences by the simultaneous primer extension of complementary strands of DNA. In other words, PCR is a reaction for making multiple copies or replicates of a target nucleic acid flanked by primer binding sites, such reaction comprising one or more repetitions of the following steps: (i) denaturing the target nucleic acid, (ii) annealing primers to the primer binding sites, and (iii) extending the primers by a nucleic acid polymerase in the presence of nucleoside triphosphates. Usually, the reaction is cycled through different temperatures optimized for each step in a thermal cycler instrument. Particular temperatures, durations at each step, and rates of change between steps depend on many factors. For example, in a conventional PCR using Taq DNA polymerase, a double stranded target nucleic acid may be denatured at a temperature >90° C., primers annealed at a temperature in the range 50-75° C., and primers extended at a temperature in the range 72-78° C. The term “PCR” encompasses derivative forms of the reaction, including but not limited to, RT-PCR, real-time PCR, nested PCR, quantitative PCR, multiplexed PCR, and the like. Reaction volumes range from a few hundred nanoliters, e.g. 200 nL, to a few hundred μL, e.g. 200 μL. “Reverse transcription PCR,” or “RT-PCR,” means a PCR that is preceded by a reverse transcription reaction that converts a target RNA to a complementary single stranded DNA, which is then amplified. “Real-time PCR” means a PCR for which the amount of reaction product, i.e. amplicon, is monitored as the reaction proceeds. There are many forms of real-time PCR that differ mainly in the detection chemistries used for monitoring the reaction product. “Nested PCR” means a two-stage PCR wherein the amplicon of a first, PCR becomes the sample for a second PCR using a new set of primers, at least one of which binds to an interior location of the first amplicon. As used herein, “initial primers” in reference to a nested amplification reaction mean the primers used to generate a first amplicon, and “secondary primers” mean the one or more primers used to generate a second, or nested, amplicon. “Multiplexed PCR” means a PCR wherein multiple target sequences (or a single target sequence and one or more reference sequences) are simultaneously carried out in the same reaction mixture. Usually, distinct sets of primers are employed for each sequence being amplified. “Quantitative PCR” means a PCR designed to measure the abundance of one or more specific target sequences in a sample or specimen. Quantitative PCR includes both absolute quantitation and relative quantitation of such target sequences. Quantitative measurements are made using one or more reference sequences that may be assayed separately or together with a target sequence. The reference sequence may be endogenous or exogenous to a sample or specimen, and in the latter case, may comprise one or more competitor templates. Typical endogenous reference sequences include segments of transcripts of the following genes: β-actin, GAPDH, β2-microglobulin, ribosomal RNA, and the like.

“Readout” means a parameter, or parameters, which are measured and/or detected that can be converted to a number or value. In some contexts, a readout may refer to an actual numerical representation of such collected or recorded data. For example, a readout of fluorescent intensity signals from a microarray is the address and fluorescence intensity of a signal being generated at each hybridization site of the microarray; thus, such a readout may be registered or stored in various ways, for example, as an image of the microarray, as a table of numbers, or the like.

“Sample” (or “biological sample” which is used synonymously) means a quantity of material from a biological, environmental, medical, or patient source in which detection or measurement of target biomolecule, such as a target nucleic acids, is sought. On the one hand it is meant to include a specimen or culture (e.g., microbiological cultures). On the other hand, it is meant to include both biological and environmental samples. A sample may include a specimen of synthetic origin. Biological samples may be animal, including human, fluid, solid (e.g., stool) or tissue, such as, fluids from nasal or other swabs, as well as liquid and solid food and feed products and ingredients such as dairy items, vegetables, meat and meat by-products, and waste. Biological samples may include materials taken from a patient including, but not limited to cultures, blood, saliva, tears, sweat, urine, cerebral spinal fluid, pleural fluid, milk, lymph, sputum, semen, needle aspirates, and the like. Environmental samples include environmental material such as surface matter, soil, water and industrial samples, as well as samples obtained from food and dairy processing instruments, apparatus, equipment, utensils, disposable and non-disposable items. These examples are not to be construed as limiting the sample types applicable to the present invention.

### DETAILED DESCRIPTION OF THE DISCLOSURE

The present disclosure is directed to methods, systems and self-metering cartridges, including microfluidic devices, for implementing rapid low-cost point-of-care bioassays, including nucleic acid-based bioassays. In one embodiment, the disclosure describes a cartridge for performing a multiplexed bioassay on a biological sample to determine the presence or quantity of one or more biomolecules, such as one or more polynucleotides. The cartridge may be associated with an appliance that is a multi-use device that provide one or more thermal sources, negative and positive pressure sources, light sources, optical detectors, optical filters, and optical occluders. In some embodiments, the cartridge comprises a lysis buffer chamber (e.g., the blister pack or blister chamber), a sample chamber, a first detection chamber, and a second detection chamber.

In some embodiments, the method of operating the system comprises amplifying a biomolecule, and associating the amplified biomolecule with a fluorescent label that is capable of optical detection, such as a fluorophore. In some embodiments, the fluorescent label can be covalently or non-covalently bound to the amplified biomolecule. In some embodiments, the fluorescent label comprises a fluorophore. Thus, in certain embodiments, the method comprises the isolation of at least one amplified biomolecule comprising at least one fluorophore. For example, in certain embodiments it can be said that the at least one amplified biomolecule comprises fluorophore 1A, wherein fluorophore 1A is specific to said amplified biomolecule. In some embodiments, a second amplified biomolecule comprises fluorophore 1B, wherein fluorophore 1B is specific to said second amplified biomolecule. Thus, in certain embodiments, the method comprises the use of multiple fluorophores that are different and, thus, capable of emitting different light signals for optical detection and identification of different biomolecules (i.e., multiplexed detection). However, in some embodiments, fluorophores 1A and 1B can be the same and, thus, are associated with the same type of amplified biomolecule, wherein the use of the same fluorophore in different detection chambers can permit redundancy in the testing for the amplified biomolecule. In other embodiments, fluorophores 1A and 1B can be the same but associated with different probes located in each of the first and second detection chambers, wherein the use of the same probe in different detection chambers can be used to detected different biomolecules.

Thus, in some embodiments, the method comprises isolating a first amplified biomolecule in a first detection chamber, wherein the first amplified biomolecule comprises fluorophore 1A; isolating a second amplified biomolecule in a second detection chamber, wherein the second amplified biomolecule comprises fluorophore 2A; illuminating the first detection chamber with a light source, wherein illuminating said first detection chamber induces fluorophore 1A of the first amplified biomolecule to produce emitted light signal 1A; and illuminating the second detection chamber with a light source, wherein illuminating said second detection chamber induces fluorophore 2A of the second amplified biomolecule to produce emitted light signal 2A. In some embodiments, the light source that illuminates the first and second detection chambers is the same. In some embodiments, the light source that illuminates the first detection chamber is different form the light source that illuminates the second detection chamber.

In some embodiments, the light source emits at least one wavelength of light, which is directed towards the first and/or second detection chambers. In certain embodiments, the light source may comprise a light-emitting diode (LED), which provides a broad spectrum of light wavelengths. Thus, in some embodiments, the light from the light source may be filtered to provide a narrower band of wavelengths. In some embodiments, the light source may comprise a laser, which comprises a smaller band of wavelengths. In such embodiments, filtering the laser light source may not be necessary.

In one embodiment, the method further comprising positioning an optical occluder in a first position to block the second detection chamber from at least a portion of the first wavelength of light, wherein the first detection chamber is exposed to the first wavelength of light and fluorophore 1A of the first biomolecule produces emitted light signal 1A. In one embodiment, the method further comprises positioning the optical occluder in a second position to block the first detection chamber from at least a portion of the first wavelength of light, wherein the second detection chamber is exposed to the first wavelength of light and fluorophore 2A of the second biomolecule produces emitted light signal 2A. In some embodiments, emitted light signals 1A and/or 1B are filtered to produce at least one filtered light signal that is detected by the optical detector.

Applicant has surprisingly discovered that the use of an optical occluder to shield the first detection chamber while the second detection chamber is being exposed to wavelength(s) of light from a light source (and vice-versa) permit efficient isolation of different signals being sourced from the same biological sample. For example, amplifying two or more amplified biomolecules (e.g., polynucleotides) from the same sample requires the use of different fluorescent labels (e.g., fluorophores) for accurate detection for each respective biomolecule. However, if both amplified biomolecules are exposed to the same light source in the same detection chamber, a plurality of emitted light signals from said fluorescent labels can complicate the accurate detection of the respective biomolecules by an optical detector. However, if amplified biomolecules are associated with reagents and fluorescent labels in separate detection chambers that exposed to optical detection at different times, two or more amplified biomolecules can be detected using one biological sample disposed in a single cartridge. Using an optical occluder to block one detection chamber while the second detection chamber is being exposed to wavelengths of light thus permits the detection of two or more amplified biomolecules using a single cartridge and a single appliance having one light source and one optical detector.

As used herein, the term “optical occluder” means any piece of equipment or hardware that is capable of blocking one detection chamber from at least a portion of the wavelengths being emitted from a light source while allowing another detection chamber to be exposed to said wavelengths at the same time. In some embodiments, the optical occluder is capable of being moved from one position to another to switch the detection chambers that are being exposed to the wavelengths produced by the light source. In some embodiments, the optical occluder is capable of being moved from one position to another to switch the detection chambers that are being detected by the detector. In some embodiments, the optical occluder may be “slid” or “rotated” from a first position to second position to permit such operation. In other embodiments, the optical occluder may be stationary, such that the cartridge itself is repositioned through a mechanism in the appliance that causes a previously-blocked detection chamber to be exposed to the light source or to the optical detector.

As used herein, the term “bioassay” or “assay” means any assay to detect or measure the quantity of a biomolecule. Exemplary biomolecules that may be detected or measured include deoxyribonucleic acids (DNAs), ribonucleic acids (RNAs), proteins, peptides, polysaccharides, lipids, and the like. Further exemplary biomolecules include genes, gene fragments, messenger RNAs (mRNAs), hormones, vitamins, enzymes, antibodies, antigens, coenzymes, immunoglobulins, and the like (e.g., Lehninger, Biochemistry, 2^{nd} Edition (Worth Publishers, 1971)). In some embodiments, a bioassay is an assay to detect or measure the quantity of a polynucleotide. In some embodiments, a bioassay comprises a polynucleotide amplification. In some embodiments, such bioassays that include a polynucleotide amplification also include an optical readout monotonically related to the degree of amplification. In some embodiments, such optical readout is a fluorescent signal. In some embodiments, such bioassays that include a polynucleotide amplification carry out an isothermal polynucleotide amplification.

In one aspect, the embodiments of the disclosure provide for cartridges that are designed to (i) accept a biological sample, (ii) contain lysis buffers that release biomolecules of interest from the biological sample, and (iii) contain assay reagents to mix with the released biomolecules and to amplify the target biomolecules to detect the presence or a quantity of said target biomolecules. In some embodiments, the cartridges described herein use gravity to redistribute a released lysis buffer in the cartridges, which thereby exposes the biological sample to the lysis buffer. In certain embodiments, applying positive pressure to a port in fluid communication with the lysis buffer and biological sample causes mixing (e.g., by air bubbles produced by the applied pressure) to mix the lysis buffer and the sample. Subsequently, the mixed buffer and biological sample contained in the cartridge are driven by positive and/or negative pressure from the appliance connected to the cartridge to move the materials to two or more detection chambers for amplification and detection.

In some embodiments, the cartridge comprises at least two detection chambers. In certain embodiments, the first detection chamber comprises a first set of assay reagents to produce the first amplified biomolecule. In some embodiments, the second detection chamber comprises a second set of assay reagents to produce the second amplified biomolecule. In certain embodiments, the first and/or second set of assay reagents may be included in a solid, liquid, or semi-solid form, such as one or more particles or a bead. In certain embodiments, the bead is freeze dried or lyophilized. Thus, in certain embodiments, a first lyophilized reagent bead comprises the first set of assay reagents. In certain embodiments, a second lyophilized reagent bead comprises the second set of assay reagents.

An exemplary system (e.g., cartridge and appliance) for carrying out the bioassays is also described herein. In some embodiments, the exemplary cartridge comprises: a lysis buffer chamber containing lysis buffer; a sample chamber in fluid communication with the lysis buffer chamber, a first detection chamber containing a first set of assay reagents; the first detection chamber in fluid communication with the sample chamber and a first port; and a second detection chamber containing a second set of assay reagents, the second detection chamber in fluid communication with the sample chamber and a second port.

In some embodiments, the system also comprises a pump in fluid communication with the first port and the second port, wherein the pump is capable of selectively applying a negative pressure and a positive pressure to said ports; a first light source positioned to emit a first wavelength towards the first detection chamber and the second detection chamber; an optical occluder capable of being positioned (i) in a first position to expose the first detection chamber to the first wavelength of light and block the second detection chamber from at least a portion of the first wavelength of light, and (ii) a second position to expose the second detection chamber to the first wavelength of light and block the first? detection chamber from at least a portion of the first wavelength of light; and an optical detector. In certain embodiments, the system also comprises at least one filter. In some embodiments, at least one filter can be used to filter certain wavelengths of light coming from the light source. In some embodiments, at least one filter can be used to filter the emitted light signal produced by a fluorophore (e.g., emitted light signal 1A, 1B, etc.) to produce a filtered light signal (e.g., filtered light signal 1A, 1B, etc.). In certain embodiments, each of the foregoing elements can be included in a single appliance (device) or multiple devices.

In certain embodiments, the method of operating the system comprises: introducing a biological sample into the sample chamber; allowing the lysis buffer to pass from the lysis buffer chamber to the sample chamber; allowing the lysis buffer to mix with the lysis buffer to create a biological mixture which ultimately yields a lysate of the biological sample; engaging the pump to apply a negative pressure to move a first portion of the biological mixture to the first detection chamber and a second portion of the biological mixture from the sample chamber to the second detection chamber; allowing the first portion of the biological mixture to react with the first set of assay reagents in the first detection chamber to create a first amplified biomolecule comprising fluorophore 1A; allowing the second portion of the biological mixture to react with the second set of assay reagents in the second detection chamber to create a second amplified biomolecule comprising fluorophore 2A; engaging the first light source to emit a first wavelength of light toward the first detection chamber and the second detection chamber; positioning the optical occluder in the second position to block at least a portion the second detection chamber from the first wavelength of light emitted by the first light source and expose the first detection chamber to the first wavelength of light, wherein fluorophore 1A of the first biomolecule produces emitted light signal 1A; filtering emitted light signal 1A through the first optical filter to produce filtered light signal 1A; detecting filtered light signal 1A at the optical detector; positioning the optical occluder in the first position to expose the second detection chamber to the first wavelength of light and block at least a portion of the first detection chamber from the first wavelength of light, wherein the second fluorophore of the second biomolecule produces emitted light signal 2A; filtering emitted light signal 2A from the second fluorophore through the first optical filter to produce filtered light signal 2A; and detecting filtered light signal 2A at the optical detector. In some embodiments, the system further comprises a third port in fluid communication with the sample chamber and the pump, wherein the method further comprises engaging the pump to apply a positive pressure to move air into sample chamber to mix the lysis buffer with the sample.

In some embodiments, it may be desirable to position the optical occuluder on the back side of the cartridge. In such an embodiment, the occluder will not inhibit the exposure of the detection chambers to the first wavelength of light. Instead, in this alternative embodiment, both the first and second chambers will be exposed to the first wavelength of light at the same time. However, positioning the optical occluder in a first position in the backside of the cartridge will block the emitted light signal 2A from the second detection chamber from reaching the optical filter and/or the optical detector, while permitting emitted light signal 1A from the first detection chamber to reach the detector. Then, movement of the optical occlude into a second position now blocks emitted light signal 1A from the first detection chamber while permitting emitted light signal 2A from the second detection chamber to reach the filter and/or optical detector.

In some embodiments, the system may further comprise a second wavelength. In some embodiments, the second wavelength is emitted from a second light source. In some embodiments, the second wavelength is emitted by the first light source, which may be accomplished by providing a secondary filter with the light source to produce said second wavelength. In some embodiments, the second wavelength of light is directed towards the first detection chamber and the second detection chamber, wherein the first amplified biomolecule further comprises fluorophore 1B and the second amplified biomolecule further comprises fluorophore 2B. Thus, in some embodiments, the method further comprises: engaging a light source to emit a second wavelength of light toward the first detection chamber and the second detection chamber; positioning the optical occluder in the second position to block at least a portion of the second detection chamber from the second wavelength of light, and expose the first detection chamber to the second wavelength of light to produce emitted light signal 1B; filtering emitted light signal 1B through the second optical filter to produce filtered light signal 1B; detecting filtered light signal 1B at the optical detector; positioning the optical occluder in the first position to expose the second detection chamber to second wavelength of light, and block at least a portion of the first detection chamber from the second wavelength of light, to produce emitted light signal 2B; filtering emitted light signal 2B through the second optical filter to produce filtered light signal 2B; and detecting filtered light signal 2B at the optical detector.

In some embodiments, the biological sample comprises at least one polynucleotide. In some embodiments, the first set of assay reagents is capable of replicating the at least one polynucleotide via isothermal polynucleotide amplification to produce a first amplified biomolecule. In some embodiments, the second set of assay reagents is capable of replicating the at least one polynucleotide via isothermal polynucleotide amplification to produce a second amplified biomolecule. In some embodiments, the first amplified biomolecule and second amplified biomolecule are associated with a disease, a virus, or a bacterium. In some embodiments, the first and second amplified biomolecules are the same and, e.g., can be associated with the same disease such that the use of the first and second detection chambers can be implemented to provide testing replicates for certainty of the result. In some embodiments, the first and second amplified biomolecules are different, such that the system can be used to simultaneously detect multiple diseases, viruses and/or bacteria at the same time.

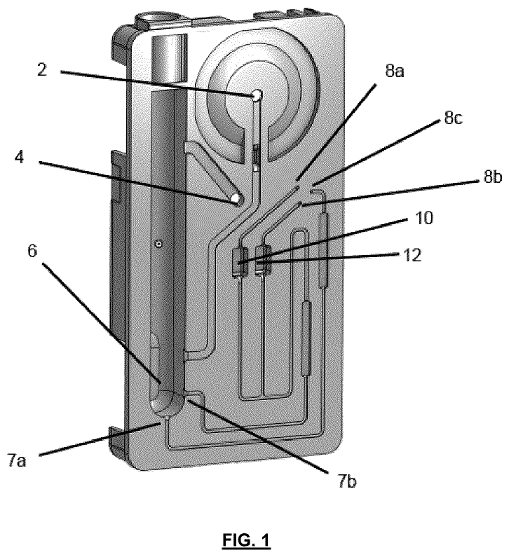

An exemplary cartridge and appliance of the disclosure for detecting a polynucleotide using, e.g., an isothermal amplification bioassay are illustrated in FIGS. 1-6. The dimensions of a cartridge depends in part on the complexity of fluidic movements required and the geometry of the samples that it is designed to accept. For example, in some embodiments multiple different bioassays may be carried out on different biomolecules from the same sample, or a single bioassay may be carried out on multiple different species of a single type of biomolecule, e.g., multiple species of DNA or RNA. Thus, a larger cartridge body may be required to accommodate multiple reagents, detection chambers, and the passages that put these elements in fluid communication. Likewise, an appliance may require more valves, pumps, thermal cycling stations, and detection stations for detecting or measuring a plurality of different biomolecule in accordance with the embodiments of the disclosure.

Exemplary cartridge body 1 of FIG. 1 exhibits a single sample chamber 6 with an opening at the top of the cartridge designed to receive a biological sample. Lysis buffer chamber 2 is in fluid communication with sample chamber 6. The cartridge body 1 may have multiple ports that establish pneumatic, optical and physical connections with an appliance. In FIG. 1, cartridge body 1 exhibits vent port 4 in fluid communication with sample chamber 6. Sample chamber 6, in turn, is in fluid communication with first detection chamber 10, second detection chamber 12, and vent ports 8a, 8b and 8c. Cartridge body 1 further comprises channel apertures 7a and 7b, which are in fluid communication via channels with vent port 8C and vent ports 8a/8b, respectively.

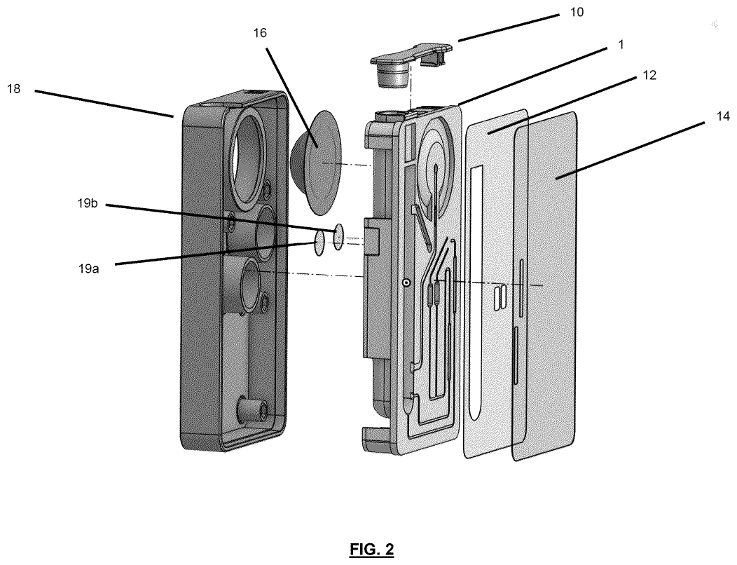

FIG. 2 represents an exploded view of cartridge 3, which comprises cartridge body 1, sample chamber cap 10, adhesive 12, and film 14 to sealingly cover the components of cartridge body 1. On the back side of cartridge body 1 is provided blister pack 16 which contains lysis buffer to engage with lysis buffer chamber 2. Hydrophobic membranes may be used with the ports to provide postive/negative pressure to the system without having liquids or samples released from the cartridge. Here, vent membrane 19a provides a seal for vent port 4, while vent membrane 19b provides a seal for both vent ports 8a and 8b. In some embodiments that further include vent port 8c, vent membrane 19b can be configured to seal vent ports 8a, 8b, and 8c. Alternatively, in some embodiments, separate seals can be provided for each vent port. Vent membrane 19a and 19b allow the passage of air but prevent the passage of liquid, allowing for the complete containment of biological materials within the cartridge without external leakage during usage.

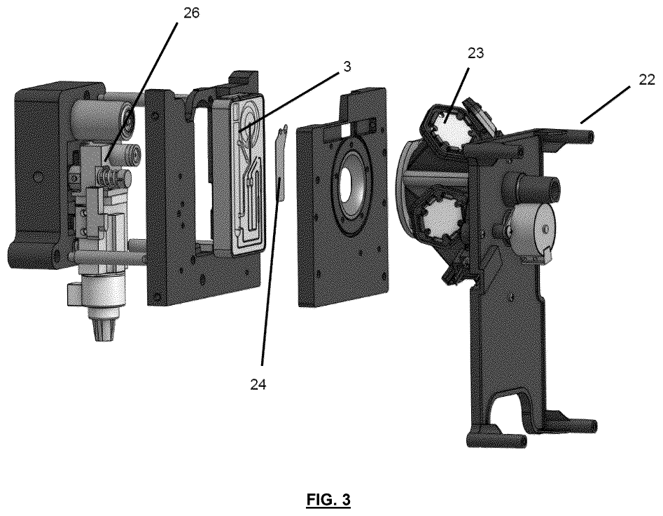

FIG. 3 provides an exploded view of an appliance with cartridge 3 for lysing, amplification and detection of a biological sample that has been received in sample chamber 6. The appliance comprises, inter alia, optics module 22, optical occluder 24, and air pump assembly 26. Optics module 22 also includes light source 23. Multiple light sources can be mounted in a circular pattern as shown in FIG. 3. One or more optical filters can be mounted on a filter wheel (not shown), which can be rotated to change filter types. Thus, a plurality of optical filters can be implemented to select one or more wavelength(s) of light (or ranges of filtered wavelengths) from the light source that will ultimately reach the first and/or second detection chambers. Vent ports 8a, 8b and 8c align with valves in air pump assembly that allow for the movement of air and biological materials within the cartridge upon the application of negative or positive pressures. In some embodiments, vent ports may align and sealingly connect with a valve having a passage that leads to a pump, a passage that leads to a vent, and a passage that leads to the cartridge. Upon mixing of the lysis buffer with the biological sample in sample chamber 6, the air pump will apply a negative pressure across vent ports 8a and 8b to move the sample into first and second detection chambers 10 and 12. There, the biological sample comes in contact with the assay reagents necessary to effect amplification and/or detection of the biomolecule.

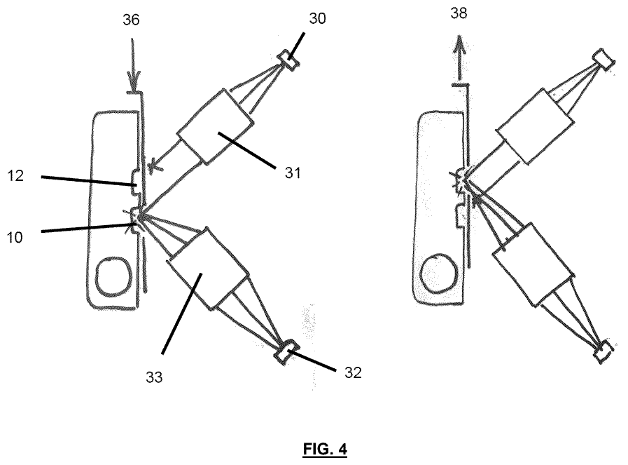

Detection of the amplified biomolecule can be accomplished as demonstrated in FIG. 4. FIG. 4 provides a top view of a cartridge engaged with the optics module of the appliance. More specifically, the amplified biomolecules comprising fluorophore(s) are contained in first detection chamber 10 and second detection chamber 12. The optical occluder is placed in first position 36, which blocks second detection chamber 12 from being exposed to light emitted from light source 30 via excitation optics 31. This exposes the amplified biomolecule in detection chamber 10 to light source 30 which, in turn, emits a light signal that passes through detection optics 33 to be captured by optical detector 32. From there, the optical occluder is then moved into second position 38, which then blocks first detection chamber 10 and exposes second detection chamber 12 to light emitted from light source 30. Thus, this assembly permits the multiplexed reading of multiple biological samples in a plurality of detection chambers using a single cartridge construction.

In some embodiments, amplification of the target biomolecule may not be required for detection. For example, in some embodiments the assay may be run to detect a target protein, which may be associated with a primer and fluorophore for detection. Thus, in certain embodiments the method of detecting the target biomolecule comprises: isolating a first biomolecule in a first detection chamber, wherein the first biomolecule is bound to fluorophore 1A; isolating a second biomolecule in a second detection chamber, wherein the second amplified biomolecule is bound to fluorophore 2A; illuminating the first detection chamber with a light source, wherein illuminating said first detection chamber induces fluorophore 1A of the first biomolecule produce emitted light signal 1A; and illuminating the second detection chamber with a light source, wherein illuminating said second detection chamber induces fluorophore 2A of the second biomolecule to produce emitted light signal 2A.

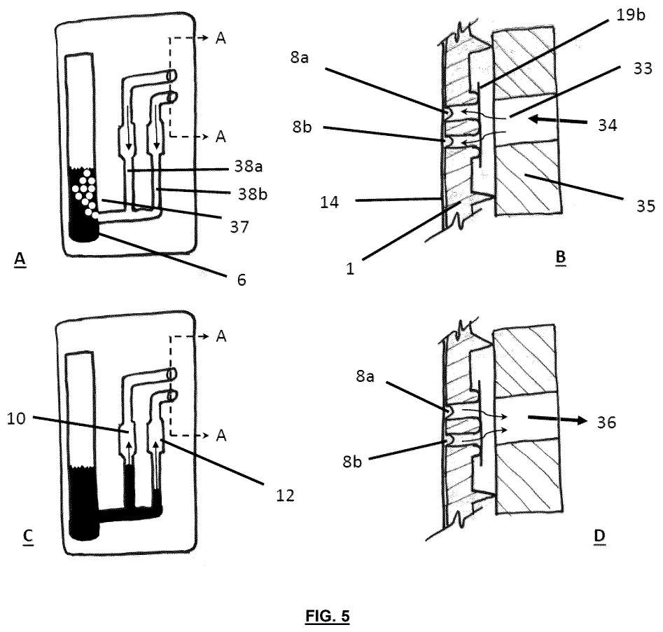

FIG. 5 provides for multiple embodiments in which the biological sample in sample chamber 6 is mixed and then drawn into the detection chambers via positive pressure and negative pressure, respectively. More specifically, Panel A in FIG. 5 demonstrates embodiments of a cartridge comprising the application of positive pressure across vent ports 8a and 8b to push air down through the system via passages 38a and 38b to generate air bubbles 37, which mix the biological sample and lysis buffer in sample chamber 6. Panel B represents a more detailed look at this with a cross-sectional view (indicated by dashed arrows A in Panel A) of cartridge 1 with back film 14 and hydrophobic vent membrane 19b. The appliance provides a sealed connection to cartridge 1 via seal 35, which in turn defines aperture 33 through which positive air pressure 34 is applied across vent ports 8a and 8b, wherein the arrows represent the direction of pressure applied. Once mixing is complete, panel C shows the application of a negative pressure across vent ports 8a and 8b that then pulls the biological sample towards first detection chamber 10 and second detection chamber 12. At the appliance level, this is also shown in the cross-sectional view (indicated by dashed arrows A in Panel C) of Panel D in which negative pressure 36 is applied across both vent ports 8a and 8b, wherein the arrows represent the direction of pressure applied.

In an alternative to the embodiment provided in FIG. 5, the cartridge body may comprise a structure that is akin to that provided in FIG. 1. In such an embodiment, vent port 8c may be added to the cartridge design for a variety of different reasons. For example, placement of channel aperture 7a at the bottom of sample chamber 6 permits the entry of bubbles from the bottom of sample chamber 6 to effectively mix the biological sample and the lysis buffer. Additionally, in some embodiments, the sediments present in the sample chamber (presented by the biological sample and/or lysis buffer) that ultimately settle to the bottom of the sample chamber can clog the channels. Thus, placement of channel aperture 7b on the side of the sample chamber just above the bottom of the sample chamber permits the movement of the lysed biological sample into the channel and up to the detection chambers when sediment has settled at the bottom. Accordingly, the implementation of vent ports 8a, 8b and 8c, along with the placement of channel apertures 7a and 7b, can permit the effective mixing of materials in the sample chamber and movement of the lysed biological sample into the detection chambers without concern of channel clogging.

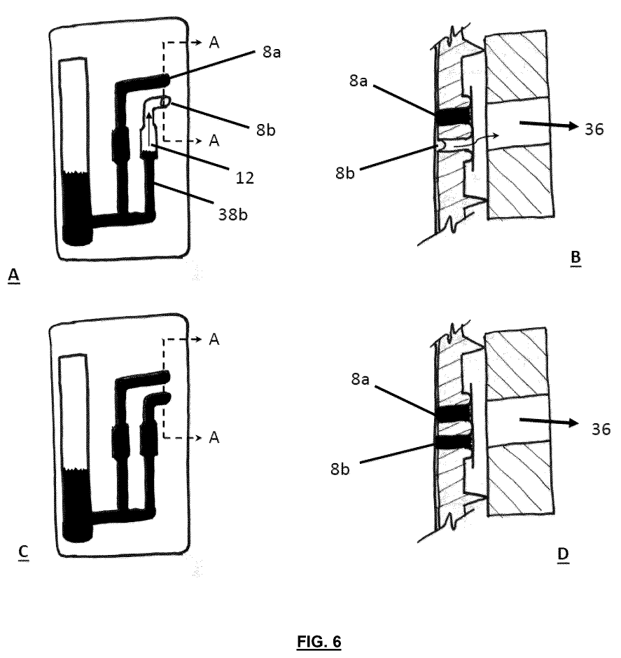

FIG. 6 demonstrates the movement of biological samples into the detection chambers. More specifically, Panel A demonstrates the application of a negative pressure across vent ports 8a and 8b results in the movement of biological sample into, and through, the first detection chamber until the fluid is stopped at vent port 8a by vent membrane 19b. As shown in Panel B, once the fluid is stopped by vent membrane 19b, the negative pressure 36 is still applied across vent port 8b. This, in turn, results in the continued advancement of biological sample through passage 38b and into second detection chamber 12. This ultimately results in the filling of both detection chambers as shown in Panel C, with the biological sample no longer being advanced once the fluid is stopped by vent membrane 19b, as seen in Panel D. In some embodiments, the advancement of biological sample to ports 8a and 8b can be aided by opening (venting) vent port 4.

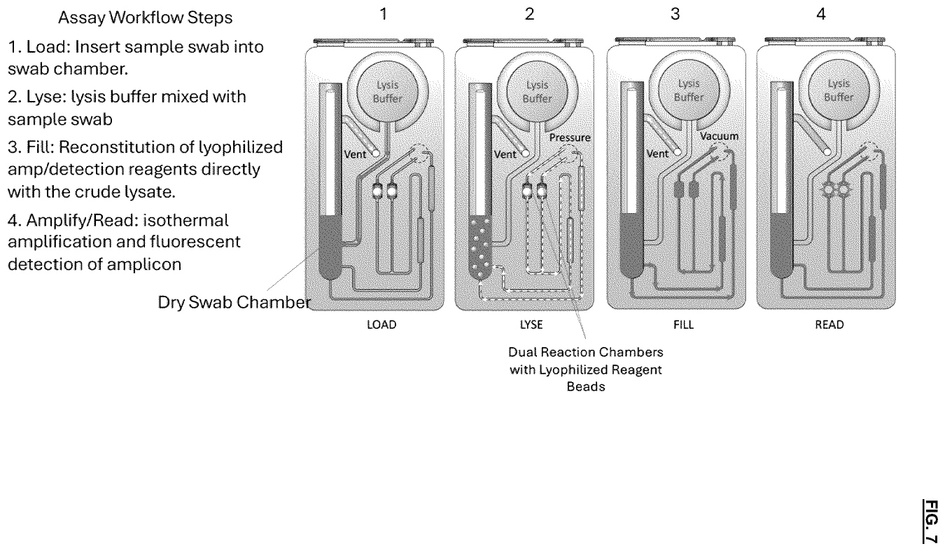

FIG. 7 provides an assay workflow for an exemplary diagnostic cartridge. In this embodiment, it is demonstrated that positive pressure can be applied against vent ports 8a, 8b and 8c (see FIG. 1), while allowing vent port 4 to remain open, to provide bubbling in the sample chamber to allow for mixing of the biological sample and the lysis buffer (step 2). From there, application of negative pressure against all three vent ports 8a, 8b and 8c allows for movement of the reaction mixture into the detection chambers for reconstitution of the lyophilized bioassay reagents (step 3). In some embodiments, it has been shown that this proprietary system design allows for the use of an appliance with a single pump that can apply negative and positive pressure across multiple vent ports at the same time. Finally, amplification of the biomolecule and detection of the associated fluorophore can be accomplished by the heat source and optical detection hardware housed within the appliance (step 4).

In some embodiments, an appliance may also include a heating or thermal control component for maintaining a detection chamber at a predetermined temperature, e.g., for an isothermal amplification assay, or for cycling a detection chamber among several temperatures, e.g., for performing a polymerase chain reaction. In embodiments employing an isothermal bioassay, a predetermined temperature may be in the range of from 55° C. to 70° C. is employed, and in some embodiments, a predetermined temperature may be in the range of from 60° C. to 65° C. is employed. Blister pouches may be used with the present disclosure. This configuration allows lysis buffer to move into the sample chamber where it contacts sample for an incubation period. In one embodiment, during the incubation period heat may also be applied to sample chamber to help release the biomolecules of interest.

In some embodiments, a predetermined incubation period depends on the nature of the sample and lysis reagents used. In some embodiments, a predetermined incubation period or time is in the range of from 1 min to 30 min, or in the range of from 2 min to 15 min. Also, in some embodiments, one or more, or all, bioassay reagents may be stored in a blister pouch that releases the bioassay reagents by mechanical actuation, similarly to the lysis buffer. However, in other embodiments the bioassay reagents are contained in one or both of the detection chambers.

In some embodiments, the vent ports described herein may be operationally associated with a pump or pressure source so that pressure is applied to the column of lysis buffer to force it back into a sample chamber to provide mixing and incubation of lysis buffer with biological sample from, e.g., a swab. The vent ports may be operationally associated with a pump that generates pressure by moving a piston in a predetermined amount, upon receiving an actuation signal from the control system of the appliance. In one embodiment, the pumps may be precision piston-style pumps, e.g. Idex Health & Science (Lake Forest, IL); Peri-Pump (Takasago Fluidics Systems, Westborough, MA); or the like. Other types of pumps, e.g., diaphragm, and other pressure sources may be employed with the embodiments of the disclosure.

In some embodiments, the bioassay reagents may be held in a separate reagent chamber, or there may be multiple reagent chambers that hold different components for a bioassay, for example, primers in one compartment and polymerase in another compartment. Such compartments may be arranged in serial fashion, so that the components are stored separately, but that an application of pressure forces all components to flow through the same passage to the detection chamber(s). Alternatively, multiple components can each be stored separately in parallel branches each with a single reagent chamber. In another embodiment, bioassay reagents may be delivered independently to one or both of the detection chambers.

In some embodiments, when the biomolecule of interest is a polynucleotide and its detection is based on an isothermal reaction, a heater, or thermal source, in the associated appliance, is actuated to maintain the detection chamber(s) at a predetermined temperature for the isothermal amplification. After a predetermined time for the isothermal reaction to run, or after it runs to completion, a measurement is made with a detection station of the appliance. Reaction times may vary widely depending on the bioassay employed. For conventional isothermal bioassays, such as loop-mediated isothermal amplification (LAMP), predetermined times from a reaction to run is in the range of from 5 to 30 min, or in the range of from 10 to 30 min. In some embodiments, signals of a bioassay may be collected over a period of from 0 to 30 min, or a period from 2 to 30 min. When an isothermal amplification assay releases fluorescent molecules, for example, in proportion to the amount of biomolecule of interest in the reaction mixture, then a detection station may comprise an excitation beam, e.g., a laser diode of an appropriate frequency, and an optical detector (e.g., fluorometer), and the readout of the assay may be a fluorescence intensity.

In certain embodiments, exemplary assay reagents are illustrated for an isothermal amplification reaction: primers, nucleoside triphosphates, and polymerase. In an exemplary embodiment, after the biological sample has been incubated in lysis buffer released by the blister pack, the lysis buffer containing released polynucleotides is moved through the channels to the detection chambers. Upon reaching the detection chambers, the lysis buffer/polynucleotide mixture will re-hydrate dried reagents upon heating and mixing.

In some embodiments, for example, where target biomolecules are nucleic acids, such as RNA or DNA, lysis buffers may include additional components to protect target RNAs or DNAs from degradation after extraction by the lysis buffer. In particular, a lysis buffer may contain or be mixed with nuclease inhibitors that are designed to inactivate nucleases that may be released along with target RNAs or DNAs by a lysis buffer. Nuclease inhibitors may have a variety of compositions, e.g. antibodies, organic polymers, and the like. In some embodiments, lysis buffer and nuclease inhibitors are stored separately, such that a lysis buffer when released (e.g. by puncturing a blister pouch), in turn, releases the nuclease inhibitor from a storage chamber as it flows to the sample chamber. The nature of the nuclease inhibitor employed is a factor in how it is stored in a cartridge. In some embodiments, nuclease inhibitors may be stored in a porous material that a lysis buffer flows through after puncturing a blister pouch. In this manner, as the lysis buffer releases target RNAs or DNAs from the sample, nuclease inhibitors will be present to prevent degradation of the target RNAs or DNAs. An exemplary porous material for storing a nuclease inhibitor is porous material is a polyethersulfone (PES) frit, e.g. available from Porex Corporation.

As mentioned above, cartridges of the present disclosure may be used with a variety of assays for biomolecules, including polymerase chain reactions (PCRs), although such applications may require additional chambers and corresponding modifications to an appliance.

In some embodiments, it may be desirable to measure the presence of at least two polynucleotides, for example, a target polynucleotide and a control, or internal standard, polynucleotide. The latter polynucleotide may be internal, or indogenous, to the sample or it may be an external, or an exogenous molecule that is added to the sample in a predetermined quantity. For example, an internal standard polynucleotide may be pre-loaded into the sample chamber as a dried reagent that is re-hydrated upon exposure to the lysis buffer. Alternatively, such an internal standard polynucleotide may be included with the bioassay reagents. Or, as another alternative for bioassays with polynucleotide amplification, an additional set of primers may be included with the assay reagents for amplifying an internal standard indogenous to the sample. In some embodiments, by a relative signal generated by the internal standard and an analyte polynucleotide, a quantity of analyte polynucleotide may be estimated.

Two (or more) sets of detection chamber may be fabricated in a single cartridge for detecting or measuring two polynucleotides of interested. Various embodiments for detecting a larger plurality of target polynucleotides may be fabricated using the same principle of operation as cartridges designed for detecting a single or two polynucleotides. After the sample is inserted and the lysis buffer release, it flows for mixture of the biological sample in the sample chamber. From there, the lysed mixture then flows into a first passage by application of positive/negative pressure applied to the vent ports. After mixing in the sample chamber, negative pressure is applied to the vent ports in fluid communication with the detection chambers to pull the reaction mixtures into the detection chambers.

The exemplary cartridges described herein, and the elements described above, such as, chambers and passages, may be formed in, a wide variety of materials well-known in the microfluidics field, such as, silicon, glass, plastic, or the like, e.g. Ren et al, Acc. Chem. Res., 46(11): 2396-2406 (2013). In some embodiments, the planar body (202) of the cartridge comprises a plastic, such as, polystyrene, polyethylenetetraphthalate glycol, polyethylene terephthalate, polymethylmethacrylate, polyvinylchloride, polycarbonate, thermo plastic elastomer or the like. Devices disclosed herein may be fabricated with or in plastic using well-known techniques including, but not limited to, hot embossing, injection molding, laser cutting, milling, etching, 3D printing, or the like. Appliances for use with cartridges of the present disclosure may be constructed using conventional engineering design principles and materials, e.g., as described in Moore (cited above). For example, the cartridges may be produced by injection molding techniques to produce the sample chamber, connecting lysis buffer chamber, passages, detection chambers, and vent ports. The blister pouch may be inserted into the cavity of the lysis buffer, and hydrophobic membranes and elastic layers may be inserted into the port cavities to form a liquid barrier and mechanism for opening and closing the vent ports, for example, as disclosed by Chen et al, Biomed. Microdevices, 12(4): 705-719 (2010).

As noted above, the planar body of the cartridge may be fabricated using conventional injection molding techniques. From there, the cartridge may be assembled by adhering a cover and/or film to the front side of the cartridge body, and a cover on the obverse side of planar body with insertion of a blister pouch. The above part may be assembled using conventional adhesives.

A wide variety of bioassays may be performed in cartridges designed and manufactured in accordance with the present disclosure. In some embodiments, bioassays implemented herein may be nucleic acid assays, and particularly nucleic acid assays that employ isothermal amplification of one or more target polynucleotides. In some embodiments, isothermal amplification may be advantageous because the added components required for thermal cycling, possibly in a separate chamber, are avoided. Many isothermal amplification techniques may be used including, but not limited to, Nucleic acid sequence-based amplification (NASBA), transcription mediated amplification (TMA), self-sustained sequence replication (3SR), signal-mediated amplification of RNA technology (SMART), strand displacement amplification (SDA), rolling circle amplification (RCA), loop-mediated isothermal amplification of DNA (LAMP), isothermal multiple displacement amplification (TMDA), helicase dependent amplification (HDA), single primer isothermal amplification (SPIA), circular helicase-dependent amplification (cHDA), recombinase polymerase amplification (RPA) and the like. In one embodiment, cartridges described herein perform a LAMP isothermal amplification. In such embodiments, one or more reagent chambers comprise a DNA polymerase, a primer set for a target polynucleotide, and deoxynucleoside triphosphates (dNTPs). In a further embodiment for detecting RNA target biomolecules, the one or more reagent chambers further contain a reverse transcriptase. In some embodiments, a LAMP amplification product is detected optically. In some embodiments, such optical detection is based on an optical measure of the turbidity of the LAMP amplification mixture, e.g. magnitude of light transmission, magnitude of light scatter, or the like, Zhu et al, ACS Omega, 5: 5421-5428 (2020). In other embodiments, a LAMP amplification product is detected colorimetrically, e.g., Goto et al, Biotechniques, 46(3): 167-172 (2009). In still other embodiments, a LAMP amplification product is measured by fluorescence, e.g., Gadkar et al, Scientific Reports, 8:5548 (2018); Hardinge et al, Scientific Reports, 9:7400 (2019); or the like, wherein fluorescence intensity may be monotonically related to amount of target polynucleotide in a sample. In some embodiments, an intercalating fluorescent DNA dye is used to measure the quantity of LAMP amplification product, e.g,. Oscorbin et al, Biotechniques, 61(1): 20-25 (2016); Quyen et al, Frontiers Microbiol., 10: 2234 (2019); or the like.

The exemplary cartridges described herein, and the elements described above, such as, chambers and passages, may be formed in a wide variety of materials well-known in the microfluidics field, such as, silicon, glass, plastic, or the like, e.g. Ren et al, Acc. Chem. Res., 46(11): 2396-2406 (2013). In some embodiments, the planar body (202) of the cartridge comprises a plastic, such as, polystyrene, polyethylenetetraphthalate glycol, polyethylene terephthalate, polymethylmethacrylate, polyvinylchloride, polycarbonate, thermoplastic elastomer or the like. Devices disclosed herein may be fabricated with or in plastic using well-known techniques including, but not limited to, hot embossing, injection molding, laser cutting, milling, etching, 3D printing, or the like. Appliances for use with cartridges of the present disclosure may be constructed using conventional engineering design principles and materials, e.g., as described in Moore (cited above). For example, the cartridges may be produced by injection molding techniques to produce the sample chamber, connecting lysis buffer chamber, passages, detection chambers, and vent ports. The blister pouch may be inserted into the cavity of the lysis buffer, and hydrophobic membranes and elastic layers may be inserted into the port cavities to form a liquid barrier and mechanism for opening and closing the vent ports, for example, as disclosed by Chen et al, Biomed. Microdevices, 12(4): 705-719 (2010).

Cartridges and appliances of the present disclosure may be adapted to detect and measure biomolecules in a wide variety of biological samples. Typically, a lysis buffer or lysis condition is selected to facilitate access of the biomolecules of interest in a sample to reagents of a bioassay. Lysis may be accomplished or facilitated mechanically, chemically, electrically or thermally. Determining the best lysis buffer for a particular sample type and biomolecule can be accomplished. In some embodiments, a reaction mixture for a bioassay may comprise a lysis buffer to facilitate access of the assay reagents to target nucleic acids. Lysing conditions may vary widely and may be based on the action of heat, detergent, protease, alkaline, chaotropic agents or combinations of such factors.

Whenever biomolecules of interest are viral polynucleotides in a sample comprising viral particles shed into a biological fluid, e.g., saliva, in some embodiments, a lysis buffer may comprise agents to disrupt the viral protein coat and to protect the release nucleic acids, such as RNA. In some embodiments, a lysis buffer may comprise a chaotropic agent, a detergent and a nuclease inhibitor. Exemplary chaotropic agents include guanidinium thiocyanate and guanidinium chloride. Exemplary lysis buffers for use with RNA viruses may be obtained commercially, e.g. Qiagen ATL (25-50% Guanidinium Thiocyanate (GITC) and 1-10% sodium dodecyl sulfate), VXL (25-50% GITC, 2.5-10% Triton-X-100), and AVL (50-70% GITC). A wide variety of bioassays may be performed in cartridges designed and manufactured in accordance with the present disclosure.

Cartridges and appliances of the present disclosure may be adapted to detect and measure biomolecules in a wide variety of biological samples. Typically, a lysis buffer or lysis condition is selected to facilitate access of the biomolecules of interest in a sample to reagents of a bioassay. Lysis may be accomplished or facilitated mechanically, chemically, electrically or thermally. Determining the best lysis buffer for a particular sample type and biomolecule can be accomplished. In some embodiments, a reaction mixture for a bioassay may comprise a lysis buffer to facilitate access of the assay reagents to target nucleic acids. Lysing conditions may vary widely and may be based on the action of heat, detergent, protease, alkaline, chaotropic agents or combinations of such factors. Whenever biomolecules of interest are viral polynucleotides in a sample comprising viral particles shed into a biological fluid, e.g., saliva, in some embodiments, a lysis buffer may comprise agents to disrupt the viral protein coat and to protect the release nucleic acids, such as RNA. In some embodiments, a lysis buffer may comprise a chaotropic agent, a detergent and a nuclease inhibitor. Exemplary chaotropic agents include guanidinium thiocyanate and guanidinium chloride.

Exemplary lysis buffers for use with RNA viruses may be obtained commercially, e.g. Qiagen ATL (25-50% Guanidinium Thiocyanate (GITC) and 1-10% sodium dodecyl sulfate), VXL (25-50% GITC, 2.5-10% Triton-X-100), and AVL (50-70% GITC).

Consistent with the above disclosure, the examples of systems and methods enumerated in the following clauses are specifically contemplated and are intended as a non-limiting set of examples.
Clause 1. A method including isolating a first biomolecule in a first detection chamber, wherein the first biomolecule is bound to fluorophore 1A; isolating a second biomolecule in a second detection chamber, wherein the second biomolecule is bound to fluorophore 2A; illuminating the first detection chamber with a first light source, wherein illuminating said first detection chamber induces the fluorophore 1A of the first biomolecule to produce an emitted light signal 1A; and illuminating the second detection chamber with a second light source, wherein illuminating said second detection chamber induces the fluorophore 2A of the second a biomolecule to produce an emitted light signal 2A.
Clause 2. The method of any foregoing clause, further including filtering the emitted light signal 1A through a filter to produce a filtered light signal 1A, and detecting the filtered light signal 1A with an optical detector.
Clause 3. The method of any foregoing clause, further including filtering the emitted light signal 2A through a filter to produce a filtered light signal 2A, and detecting the filtered light signal 2A with an optical detector.
Clause 4. The method of any foregoing clause, where the filtered light signal 1A and the filtered light signal 2A are detected by the optical detector.
Clause 5. The method of any foregoing clause, where the first light source that illuminates the first detection chamber and the second light source that illuminates the second detection chamber are a same light source.
Clause 6. The method of any foregoing clause, where the same light source emits a first wavelength of light.
Clause 7. The method of any foregoing clause, where the first wavelength of light is directed towards the first detection chamber and the second detection chamber.
Clause 8. The method of any foregoing clause, further including positioning an optical occluder in a first position to block the second detection chamber from at least a portion of the first wavelength of light, where the first detection chamber is exposed to the first wavelength of light and the fluorophore 1A of the first biomolecule produces the emitted light signal 1A.
Clause 9. The method of any foregoing clause, further including positioning an optical occluder in a second position to block the first detection chamber from at least a portion of the first wavelength of light, where the second detection chamber is exposed to the first wavelength of light and the fluorophore 2A of the second biomolecule produces the emitted light signal 2A.
Clause 10. The method of any foregoing clause, further including a third light source that emits a second wavelength of light.
Clause 11. The method of any foregoing clause, where the first biomolecule is further bound to fluorophore 1B.
Clause 12. The method of any foregoing clause, where the second biomolecule is further bound to fluorophore 2B.
Clause 13. The method of any foregoing clause, further including illuminating the first detection chamber with the third light source, where illuminating said first detection chamber induces the fluorophore 1B of the first biomolecule produce an emitted light signal 1B.
Clause 14. The method of any foregoing clause, further including illuminating the second detection chamber with the third light source, where illuminating said second detection chamber induces the fluorophore 2B of the second biomolecule to produce an emitted light signal 2B.
Clause 15. The method of any foregoing clause, further including filtering the emitted light signal 1B through a filter to produce a filtered light signal 1B, and detecting the filtered light signal 1B with the optical detector.
Clause 16. The method of any foregoing clause, further including filtering the emitted light signal 2B through a filter to produce a filtered light signal 2B, and detecting the filtered light signal 2B with the optical detector.
Clause 17. The method of any foregoing clause, further including isolating a biological sample in a sample chamber.
Clause 18. The method of any foregoing clause, further including transferring at least a portion of the biological sample into the first detection chamber and the second detection chamber.
Clause 19. The method of any foregoing clause, where the first detection chamber comprises a first set of assay reagents, and the second detection chamber comprises a second set of assay reagents.
Clause 20. The method of any foregoing clause, further including reacting the biological sample with the first set of assay reagents to produce the first biomolecule.
Clause 21. The method of any foregoing clause, further including reacting the biological sample with the second set of assay reagents to produce the second biomolecule.
Clause 22. The method of any foregoing clause, where the first biomolecule comprises a first amplified biomolecule, and the second biomolecule comprises a second amplified biomolecule.
Clause 23. An optical detection system including a lysis buffer chamber containing lysis buffer; a sample chamber in fluid communication with the lysis buffer chamber; a first detection chamber containing a first set of assay reagents, the first detection chamber in fluid communication with the sample chamber and a first port; a second detection chamber containing a second set of assay reagents, the second detection chamber in fluid communication with the sample chamber and a second port; a pump in fluid communication with the first port and the second port, wherein the pump is operatively configured to selectively apply a negative pressure and a positive pressure to said first port and said second port; a first light source positioned to emit a first wavelength towards the first detection chamber and the second detection chamber; an optical occluder operatively configured to be positioned in a first position to occlude the first detection chamber and expose the second detection chamber, and a second position to expose the first detection chamber and occlude the second detection chamber; a first optical filter; and an optical detector.
Clause 24. A method including introducing a biological sample into a sample chamber; allowing a lysis buffer to pass from a lysis buffer chamber to the sample chamber; allowing the lysis buffer to mix with the biological sample to create a biological mixture; engaging a pump to apply a negative pressure to move a first portion of the biological mixture to a first detection chamber and a second portion of the biological mixture from the sample chamber to a second detection chamber; allowing the first portion of the biological mixture to react with a first set of assay reagents in the first detection chamber to create a first amplified biomolecule bound to fluorophore 1A; allowing the second portion of the biological mixture to react with a second set of assay reagents in the second detection chamber to create a second amplified biomolecule bound to fluorophore 2A; engaging the first light source to emit a first wavelength of light toward the first detection chamber and the second detection chamber; positioning an optical occluder in a second position to block at least a portion the second detection chamber from the first wavelength of light emitted by the first light source, and expose the first detection chamber to the first wavelength of light, wherein fluorophore 1A of the first biomolecule produces emitted light signal 1A; filtering emitted light signal 1A from the first fluorophore through the first optical filter to produce filtered light signal 1A; detecting filtered light signal 1A at an optical detector; positioning the optical occluder to a first position to expose the second detection chamber to the first wavelength of light and block at least a portion of the first detection chamber from the first wavelength of light, wherein the second fluorophore of the second biomolecule produces emitted light signal 2A; filtering emitted light signal 2A from the second fluorophore through the first optical filter to produce filtered light signal 2A; and detecting filtered light signal 2A at the optical detector.
Clause 25. The method of any foregoing clause, where said method employs the use of the device of claim 23.
Clause 26. The method of any foregoing clause, further comprising engaging the pump to apply a positive pressure to move air into the sample chamber to mix the lysis buffer with the sample.
Clause 27. The method of any foregoing clause, further including engaging a second light source to emit a second wavelength of light toward the first detection chamber and the second detection chamber; positioning the optical occluder in the second position to block at least a portion of the second detection chamber from the second wavelength of light, and expose the first detection chamber to the second wavelength of light to produce emitted light signal 1B; filtering the emitted light signal 1B through a second optical filter to produce a filtered light signal 1B; detecting the filtered light signal 1B at the optical detector; positioning the optical occluder in the first position to expose the second detection chamber to the second wavelength of light, and block at least a portion of the first detection chamber from the second wavelength of light, to produce an emitted light signal 2B; filtering the emitted light signal 2B through the second optical filter to produce a filtered light signal 2B; and detecting the filtered light signal 2B at the optical detector.
Clause 28. The method of any foregoing clause, further including heating the sample chamber.
Clause 29. The method of any foregoing clause, further including heating the first detection chamber.
Clause 30. The method of any foregoing clause, further including heating the second detection chamber.
Clause 31. The method of any foregoing clause, where the first light source and/or the second light source comprise a light emitting diode (LED) light source.
Clause 32. The method of any foregoing clause, where the biological sample comprises at least one polynucleotide.
Clause 33. The method of any foregoing clause, where a first lyophilized reagent bead comprises the first set of assay reagents.
Clause 34. The method of any foregoing clause, where a second lyophilized reagent bead comprises the second set of assay reagents.

The above discussion is meant to be illustrative of the principles and various embodiments of the present disclosure. Numerous variations and modifications will become apparent to those skilled in the art once the above disclosure is fully appreciated. It is intended that the following claims be interpreted to embrace all such variations and modifications.

### REFERENCES

Aono et al, Japanese Patent Publ. No. JP 4-262799.
Bau et al, U.S. Patent Publ. No. 2010/0035349.
Becker et al, Talanta, 56: 267-287 (2002).
Becker-Andre et al, Nucleic Acids Research, 17: 9437-9447 (1989).
Bernard et al, Anal. Biochem., 273: 221-228 (1999).
Bjornson et al, International Patent Publ. No. WO 99/19717.
Bjornson et al, U.S. Pat. No. 6,803,019.
Buhl et al, U.S. Pat. No. 5,413,732.
Burns et al, U.S. Pat. No. 6,379,929.
Cao, “Nanostructures & Nanomaterials: Synthesis, Properties & Applications,” (Imperial College Press, London, 2004).
Cheng et al, Biochip Technology (CRC Press, 2001).
Cole, U.S. Pat. No. 5,102,788.
De Rosier et al, U.S. Pat. No. 6,294,365.
Deiman et al, Mol. Biotechnol., 20: 163-179 (2002).
Dieffenbach, editor, PCR Primer: A Laboratory Manual, 2nd Edition (Cold Spring Harbor Press, New York, 2003).
Diviacco et al, Gene, 1: 3013-3020 (1992).
E. H. Lennette (ed.), Laboratory Diagnosis of Viral Infections, second edition (Marcel Dekker, Inc., New York, 1992).
Enzelberger et al, U.S. Pat. No. 6,960,437.
Fiechtner et al, U.S. Pat. No. 10,520,498.
Fiorini et al, Biotechniques, 38(3): 429-446 (2005).
Franks et al, U.S. Pat. No. 5,098,893.
Freeman et al, Biotechniques, 26: 112-126 (1999).
Gelfand et al, U.S. Pat. No. 5,210,015.
Gill et al, Nucleosides, Nucleotides & Nucleic Acids, 27: 224-243 (2008).
Gulliksen et al, Anal. Chem., 76:9-14 (2004).
Haeberle et al, LabChip, 7: 1094-1110 (2007).
Kacian et al, U.S. Pat. No. 5,399,491,
Karami et al, J.Global Infect. Dis., 3(3): 293-302 (2011).
Kim et al, Integrative Biology, 1: 574-586 (2009).
Latham et al, U.S. Pat. No. 7,264,932.
Leone et al, Nucleic Acids Research, 26: 2150-2155 (1998),
Leone et al, Nucleic Acids Research, 26: 2150-2155 (1998).
Lizardi, U.S. Pat. No. 5,854,033.
Mackay et al, Nucleic Acids Research, 30: 1292-1305 (2002).
Maher et al, U.S. Pat. No. 6,399,952.
McMillan et al, U.S. Patent Publ. No. 2006/0068399.
McMillan, U.S. Patent Publ. No. 2006/0068398.
McPherson et al, editors, PCR: A Practical Approach and PCR2: A Practical Approach (IRL Press, Oxford, 1991 and 1995, respectively).
Mullis et al, U.S. Pat. No. 4,683,195.
Mullis et al, U.S. Pat. No. 4,683,202.
Mullis et al, U.S. Pat. No. 4,800,159.
Mullis et al, U.S. Pat. No. 4,965,188.
Nail et al (2002), Pharm. Biotechnol., 14: 281-360.
Nelson et al, U.S. Pat. No. 6,613,525.
Neyer et al, U.S. Pat. No. 6,838,156.
Notomi et al, Nucleic Acids Research, 28(12): e63 (2000).
Notomi et al, U.S. Pat. No. 6,410,278.
Pack et al, U.S. Patent Publ. No. 2008/0182312.
Polstra et al, BMC Infectious Diseases, 2: 18 (2002).
Raines et al, Patent Publ. No. 2013/0344563.
Ramsey, U.S. Pat. No. 6,033,546.
Ramsey, U.S. Pat. No. 5,858,195.
Ramsey, U.S. Pat. No. 6,001,229.
Ramsey, U.S. Pat. No. 6,010,607.
Ricco et al, International Patent Publ. No. WO 02/24322.
Sambrook et al, Molecular Cloning, Second Edition (Cold Spring Harbor Laboratory, New York, 1989).
Schaevitz et al, U.S. Pat. No. 6,908,594.
Schwegman et la (2005), Pharm. Dev. Technol., 10: 151-173.
Shen et al, U.S. Pat. No. 5,556,771.
Sia et al, Electrophoresis, 24: 3563-3576 (2003).
Smith et al, Microfluidics and Nanofluidics, 20: 163 (2016).
Smith et al, Proc. SPIE, 9705: 97050F (2016).
Soane et al, U.S. Pat. No. 5,126,022.
Soane et al, U.S. Pat. No. 6,054,034.
Soane et al, U.S. Pat. No. 6,176,962.
Svec et al, Frontiers in Oncology, 3: 1-11 (2013).
Tecott et al, U.S. Pat. No. 5,168,038.
Treml et al, U.S. Pat. No. 5,763,157.
Tyagi et al, U.S. Pat. No. 5,925,517.
Unger et al, Science, 288: 113-116 (2000).
Wang et al, U.S. Pat. No. 8,673,567.
Weusten et al, Nucleic Acids Research, 30(6) e26 (2002).
Wilding et al, U.S. Pat. No. 5,498,392.
Wilding et al, U.S. Pat. No. 5,587,128.
Wittwer et al, U.S. Pat. No. 6,174,670.
Wittwer et al, U.S. Pat. No. 6,569,627.
Zimmerman et al, Biotechniques, 21: 268-279 (1996).

What is claimed is:

1. A method comprising:
isolating a first biomolecule in a first detection chamber, wherein the first biomolecule is bound to fluorophore 1A;
isolating a second biomolecule in a second detection chamber, wherein the second biomolecule is bound to fluorophore 2A;
illuminating the first detection chamber with a first light source, wherein illuminating said first detection chamber induces the fluorophore 1A of the first biomolecule to produce an emitted light signal 1A; and
illuminating the second detection chamber with a second light source, wherein illuminating said second detection chamber induces the fluorophore 2A of the second a biomolecule to produce an emitted light signal 2A.

2. The method of claim 1, further comprising filtering the emitted light signal 1A through a filter to produce a filtered light signal 1A, and detecting the filtered light signal 1A with an optical detector.

3. The method of claim 1, further comprising filtering the emitted light signal 2A through a filter to produce a filtered light signal 2A, and detecting the filtered light signal 2A with an optical detector.

4. The method of claim 2, wherein the filtered light signal 1A and the filtered light signal 2A are detected by the optical detector.

5. The method of claim 1, wherein the first light source that illuminates the first detection chamber and the second light source that illuminates the second detection chamber are a same light source.

6. The method of claim 5, wherein the same light source emits a first wavelength of light.

7. The method of any one of claims 5, wherein the first wavelength of light is directed towards the first detection chamber and the second detection chamber.

8. The method of claim 7, further comprising positioning an optical occluder in a first position to block the second detection chamber from at least a portion of the first wavelength of light, wherein the first detection chamber is exposed to the first wavelength of light and the fluorophore 1A of the first biomolecule produces the emitted light signal 1A.

9. The method of claim 8, further comprising positioning an optical occluder in a second position to block the first detection chamber from at least a portion of the first wavelength of light, wherein the second detection chamber is exposed to the first wavelength of light and the fluorophore 2A of the second biomolecule produces the emitted light signal 2A.

10. The method of claim 1, further comprising a third light source that emits a second wavelength of light.

11. The method of claim 10, wherein the first biomolecule is further bound to fluorophore 1B.

12. The method of any of claims 10, wherein the second biomolecule is further bound to fluorophore 2B.

13. The method of claim 11, further comprising illuminating the first detection chamber with the third light source, wherein illuminating said first detection chamber induces the fluorophore 1B of the first biomolecule produce an emitted light signal 1B.

14. The method of claim 12, further comprising illuminating the second detection chamber with the third light source, wherein illuminating said second detection chamber induces the fluorophore 2B of the second biomolecule to produce an emitted light signal 2B.

15. The method of claims 13, further comprising filtering the emitted light signal 1B through a filter to produce a filtered light signal 1B, and detecting the filtered light signal 1B with the optical detector.

16. The method of claim 13, further comprising filtering the emitted light signal 2B through a filter to produce a filtered light signal 2B, and detecting the filtered light signal 2B with the optical detector.

17. The method of claim 1, further comprising isolating a biological sample in a sample chamber.

18. The method of claim 17, further comprising transferring at least a portion of the biological sample into the first detection chamber and the second detection chamber.

19. The method of claim 18, wherein the first detection chamber comprises a first set of assay reagents, and the second detection chamber comprises a second set of assay reagents.

20. The method of claim 19, further comprising reacting the biological sample with the first set of assay reagents to produce the first biomolecule.
